(19) United States
(12) Patent Application Publication
(10) Pub. No.: US 2003/0171382 A1
(43) Pub. Date: Sep. 11, 2003

(54) Pharmaceutical composition for regeneration of cirrhotic liver

(76) Inventors: Sang-Geon Kim, Seoul (KR); Keon-Wook Kang, Seoul (KR); Yoon-Gyoon Kim, Seoul (KR); Min-Kyung Cho, Seoul (KR)

Correspondence Address: CANTOR COLBURN LLP, 55 Griffin South Road, Bloomfield, CT, 06002, US

(21) Appl. No.: 10/360,698

(22) Filed: Feb. 6, 2003

(30) Foreign Application Priority Data
Feb. 9, 2002 (KR) 2002-7678

(51) Int. Cl. A61K 31/497

(52) U.S. Cl. 514/255.05

(57) ABSTRACT

The present invention provides a pharmaceutical composition and the use thereof for regeneration of liver tissues to treat cirrhotic liver, the composition including 5-(2-pyrazinyl)-4-methyl-1,2-dithiol-3-thione (oltipraz) as an active ingredient. The oltipraz composition promotes regeneration of liver tissues in a cirrhotic liver, thereby useful in treating cirrhosis.

### BACKGROUND OF THE INVENTION

1. Field of the Invention

The invention relates to a pharmaceutical composition for regenerating liver tissues in patients suffering from cirrhotic liver and a use of the composition as a regenerant of liver tissues of a cirrhotic liver.

2. Description of the Related Art

The liver plays a key role in the metabolism of xenobiotics and in the metabolism of endogenous substances. The liver is an important organ where consistent enzymatic reactions and energy metabolism occur. Among many chronic diseases in Korea, hepatitis, cirrhosis, and liver cancer are the most widespread and life threatening diseases next to cardiovascular diseases. Especially chronic drinking and binge drinking cause high likelihood of damaging the liver. The chronic liver damage resulting from viral infection or alcohol consumption is often the cause of cirrhosis or fibrosis of the liver.

Cirrhosis is a chronic liver disease with high percentage death rate and the conditions are destruction of parenchymal cells and accumulation of connective tissues. Cirrhosis is considered the most damaging among liver infections and other chronic liver diseases. Cirrhosis occurs when the damaged liver cells do not recover back to normal cells, but rather, transform into fibrous tissues such as collagen and the parenchymal cells of the liver are destroyed, resulting in the deterioration of the function and the size of the liver. Because cirrhosis may cause death in human, a development of appropriate therapeutic and preventive drugs is in high demand. However, there are no known drugs that regenerate liver cells for the treatment of cirrhosis.

Various substances, including several synthetic compounds and galenical preparations, show hepatoprotective functions both in vitro and in vivo. Although it has been known that silymarin and betaine have liver protective effects as a result of cytokine inhibition or an increase in the level of glutathione, the effects of such results are low and thus, a curative success is difficult to obtain.

It has been known that several substituents of sulfur containing dithiolthione, found naturally in cruciferous vegetables, have liver protecting effects. Among them, oltipraz, represented by Chemical Formula 1, was used in the early 1980s as a curative agent against schistosomiasis.

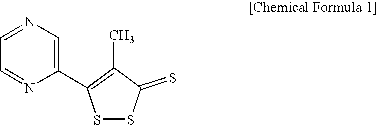

Oltipraz increases a cellular thiol content and induces expression of enzymes responsible for maintaining a glutathione (GSH) pool and detoxifying a tissue from electrophilic molecules. The activities of the following enzymes are increased by oltipraz: NAD(P)H quinone reductase, microsomal epoxide hydrolase, glutathione S-transferase (GST) and UDP glucuronyl transferase (UDP-GT). In particular, GST protects the liver from carbon tetrachloride and acetaminophen (Ansher S S, Dolan P, and Bueding E. Chemoprotective effects of two dithiolthiones and of butylhydroxyanisole against carbon tetrachloride and acetaminophen toxicity. 1983, Hepatology 3, 932-935).

Furthermore, oltipraz inhibits chemical carcinogenesis caused by benzo[a]pyrene, NDEA, and uracil mustard as well as aflatoxin B 1-induced hepatic tumorigenesis and azoxymethane-induced colon carcinogenesis (Bolton M G, Munoz A, Jacobson L P, Groopman J D, Maxuitenko Y Y, Roebuck B D, and Kensler T W. Transient intervention with oltipraz protects against aflatoxin-induced hepatic tumorigenesis. 1993, Cancer Res. 53, 3499-3504).

The known inhibitory mechanisms of carcinogenesis by oltipraz are the following. First, oltipraz increases the level of a reduced GSH, an antioxidant, in tissues. Second, it inhibits bioactivation of carcinogens by inhibiting phase I enzymes such as cytochrome P450. Third, it promotes detoxification of carcinogens by inducing phase II detoxifying enzymes including GST and UDP-GT. Fourth, oltipraz inhibits replication of the human immunodeficiency virus (HIV) type I in vitro. Fifth, it removes reactive intermediates in cells by increasing thiol levels and promotes DNA repair. It has been reported that oltipraz increases GSH levels in most tissues and removes free radicals generated by radiation or xenobiotics. It also has been known that oltipraz functions as a protective agent against radiation by helping to maintain cellular homeostasis.

Clinical trials on the chemopreventive effect of oltipraz against liver carcinogenesis have been conducted. The results showed that oltipraz is weakly active in suppressing liver carcinogenesis and that oltipraz protects the liver against toxicant-induced hepatotoxicity, at least moderately. In addition, the safety of oltipraz has been proven in toxicity studies performed in rats and dogs (Fund. Appl. Toxicol. January 1997; 35(1):9-21).

However, a chemical composition effective in regenerating liver cells of a cirrhotic liver has not yet been reported. Therefore, considering the biological function and importance of the liver in a human body, it is necessary to develop drugs that have successful curative effects in treating cirrhosis.

### SUMMARY OF THE INVENTION

The invention provides a pharmaceutical composition that is effective in regenerating liver tissues of a cirrhotic liver. In one aspect, the invention provides a composition for regenerating liver tissues of a cirrhotic liver, which comprises 5-(2-pyrazinyl)-4-methyl-1,2-dithiol-3-thione (oltipraz) as an active ingredient.

### BRIEF DESCRIPTION OF THE DRAWINGS

The features and advantages of the present invention will become more apparent by describing in detail exemplary embodiments thereof with reference to the attached drawings in which:

FIG. 1 is a graph demonstrating the increase in the survival rate of the cirrhotic rats administered with oltipraz

FIG. 2 is photographs of liver tissues of a cirrhotic rat and liver tissues after oltipraz administration and Masson's Trichrome staining.

FIG. 3a is a graph demonstrating the decrease of ascites in cirrhotic rats after oltipraz administration.

FIG. 3b is a graph demonstrating the increase of plasma albumin in cirrhotic rats after oltipraz administration.

FIG. 4a represent graphs demonstrating the increase of liver weight in cirrhotic rats after oltipraz administration.

FIG. 4b represent photographs showing that liver cell divisions are activated by oltipraz administration in cirrhotic rats.

FIG. 5a represent photographs of liver cell division and regeneration by oltipraz administration in cirrhotic rats after PCNA staining.

FIG. 5b represent photographs showing the promotion of undifferentiated stem cells into differentiated stem cells due to oltipraz administration in cirrhotic liver tissue. (Top: Thy1.1 staining, Bottom: Flt-3 staining)

FIG. 6a is a gel electrophoresis photograph showing the increase in the expression of c-Met due to oltipraz administration in cirrhotic rats.

FIG. 6b is a gel electrophoresis photograph showing increase of LAP, which is an active agent of C/EBP-β, and decrease of LIP, an inhibitory factor, and recovery of expression of C/EBP-α in cirrhotic rats after oltipraz administration.

FIG. 7a is a gel electrophoresis photograph showing a gradual increase in the amount of C/EBP-β in the nuclear fraction of the cells after incubation of hepatocytes with oltipraz.

FIG. 7b represents immunocytochemical photographs showing the translocation of C/EBP-β into the cell's nucleus when hepatocytes are incubated with oltipraz.

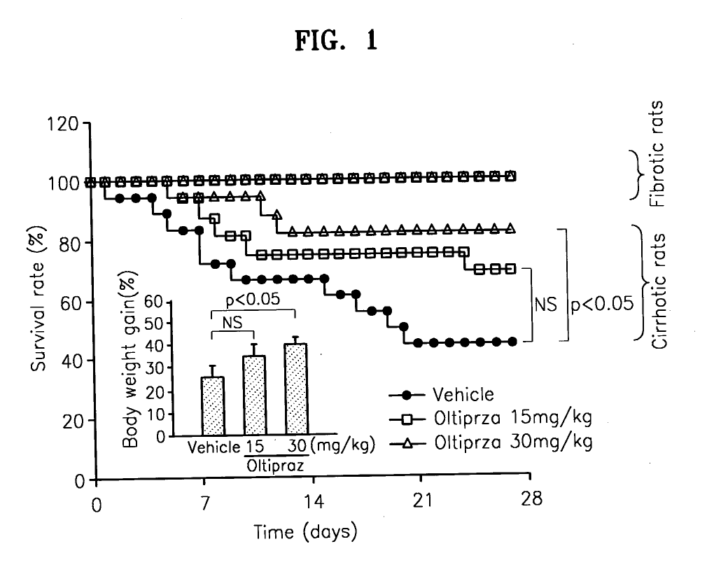

### DETAILED DESCRIPTION OF THE INVENTION

On the basis of the fact that in order to ultimately treat cirrhosis, it is not only necessary to suppress the progress of cirrhosis, but the damaged tissues must be recovered and regenerated, the inventors tried to develop a pharmaceutical composition, which has few side effects and effectively regenerates cirrhotic liver tissues, and found out that oltipraz is effective in regenerating cirrhotic liver tissues.

The regenerative ability of oltipraz for liver tissues was demonstrated by the experimental results of the invention.

In the present invention, the curative and regenerative effects of oltipraz in correcting cirrhosis and fibrosis of the liver tissues were observed in model rats that had been administered with dimethylnitrosamine (DMN) for 4 weeks for the purpose of inducing cirrhosis or liver fibrosis. The results demonstrated that although prior to administration of oltipraz, the survival rate of the rats gradually diminished, after the administration, there had been statistically significant improvement in the survival rate of the rats. Further, compared to the increased aspartate aminotransferase (AST) activity in the blood plasma of cirrhotic rats, post-oltipraz administered blood plasma indicated a lessened AST activity.

The content of albumin in the blood plasma is a representative indicator of a liver's condition and albumin is necessary to control the osmotic pressure of the blood plasma. Oltipraz in rats with cirrhotic liver significantly restored the lowered albumin level to a normal level, and further normalized the osmotic pressure of the blood plasma, thereby diminishing the ascites accompanied by liver cirrhosis.

According to the fibrosis score and Knodell score obtained by histopathological microscopic examinations of a cirrhotic liver, a large amount of fibers accumulated near the portal veins and inflamed areas were observed. However, after oltipraz administration, such liver lesions were noticeably remedied.

Besides the foregoing effects, the administration of oltipraz increased weight of a liver that was previously atrophied due to cirrhosis. The histopathological microscopic examination showed frequent liver cell divisions in a cirrhotic liver. Further, by studying the proliferating cell nuclear antigen (PCNA), which normally appears only during a period of cell growth, under a microscope after cells were stained immunochemically, oltipraz administered rats showed notable increase of the number of liver cells with PCNA. Such increase of the PCNA expression was confirmed by western blot analysis.

Further, expression of other proteins related to the proliferation of liver cells, such as c-Met, a receptor of the liver cell growth factor and CCAAT/Enhancer Binding Protein (C/EBP-β), a liver-enriched activating protein (LAP), decreased in cirrhotic rats, but were recovered in oltipraz administered rats. On the other hand, the expression of truncated isoform of C/EBP-β, a liver-enriched inhibitory protein (LIP), reduced with oltipraz administration.

When the undifferentiated stem cells of a liver were stained, cirrhotic rats showed many stem cells. However, oltipraz administered rats showed notable reduction of the stem cells in the liver. Such finding supports the inference that oltipraz induces the undifferentiated stem cells to convert to differentiated liver cells.

Accordingly, the therapeutic effect of oltipraz, an active ingredient of the composition in the present invention, is obtained due to its ability to regenerate tissues through enhanced cell division and proliferation.

When the pharmaceutical composition of the present invention is produced for actual use, the unit dosage forms suitable for oral administration, injection and the like are formulated and administered according to the conventional method adopted in the appropriate pharmaceutical field.

Appropriate oral preparation comprises a hard or soft capsule, tablet, powder, syrup, etc. The oral formulation, in addition to oltipraz as the pharmaceutically active agent, may contain one or more pharmaceutically non-active conventional carriers. For example, the oral formulation may contain excipients such as starch, lactose, carboxymethylcellulose and kaolin; binders such as water, gelatin, alcohol, glucose, arabic gum and tragacanth gum; disintegrants such as starch, dextrine and sodium alginate; and lubricants such as stearic acid, magnesium stearate and liquid paraffin.

The daily dosage of the pharmaceutical composition according to the present invention depends on various factors such as the patient's degree of liver cirrhosis, time of onset of the disease, age, health, other complications, etc. However, for the average adult, oltipraz is administered once or twice a day for a total daily dosage of 10 to 1000 mg, more preferably 50 to 300 mg. However, in cases where the patient has severe liver cirrhosis, the present invention can go beyond the scope of the above pharmaceutical composition and employ even larger dosages.

The present invention is explained in greater detail in the examples below. However, the present invention is not limited to these examples.

### EXAMPLES

Sprague-Dawley rats that were 6 weeks old and 140-160 g were used in the examples below.

### Example 1

### The Survival Rates of Rats with Cirrhosis

By continually administering rats with dimethylnitrosamine (DMN) 3 times a week for 4 weeks, test models of cirrhotic livers were achieved. At such time, the rats showing only indications of liver fibrosis were placed in a separate group from rats showing indications of cirrhosis, and the survival rates of rats with cirrhosis and rats with liver fibrosis were studied during the next 4 weeks.

The survival rate of test models with cirrhotic liver gradually declined with the passage of time and after 4 weeks, the survival rate was at 48%. The survival rate of the rats administered with 30 mg/kg of oltipraz, 3 times a week for 4 weeks, increased to 83%, indicating statistically significant improvement. Further, there was no death of rats with liver fibrosis, after the rats were administered with 30 mg/kg of oltipraz 3 times a week

### Example 2

### Amelioration of Liver Cirrhosis by Studying Tissue Samples

The histopathological effect of oltipraz on cirrhosis was studied. The liver tissue of cirrhotic rats showed significant amount of fibers accumulated around the blood vessel, forming cirrhotic nodules as a result of the buildup. When the cirrhotic rats were administered with 15 or 30 mg/kg of oltipraz, 3 times a week for 4 weeks, the accumulation of fibers was reduced in a dose-dependent manner.

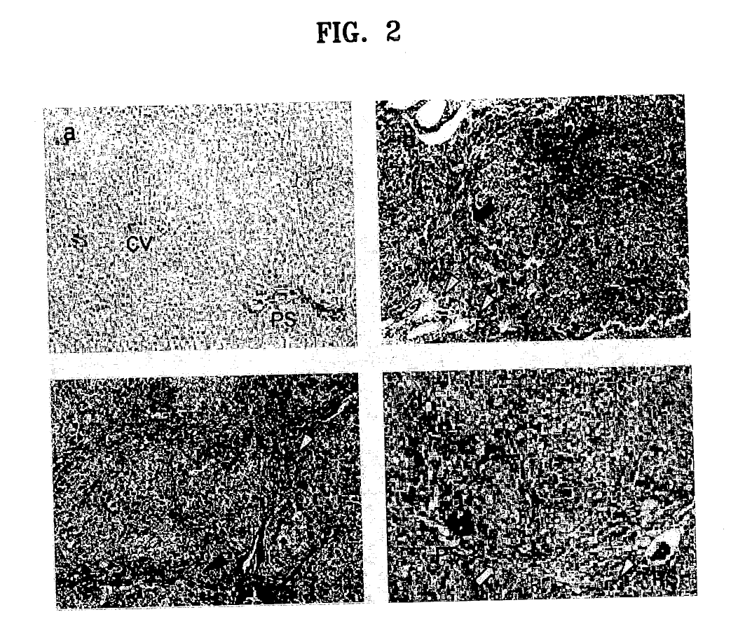

The curative effect of oltipraz on liver cirrhosis was histopathologically determined through fibrosis scores taken after Masson's trichrome staining and through Knodell scores which show the portal inflammation and the extent of fibrosis of the liver (FIG. 2, Table 1). The results show effective treatment of cirrhosis when 15 or 30 mg/kg of oltipraz were administered.

In FIG. 2, A is a photograph of a liver tissue of a normal rat, B is a photograph of liver tissue from the group having cirrhosis, C is a photograph of liver tissue from the group having cirrhosis that was administered with 15 mg/kg of oltipraz, 3 times a week for 4 weeks, and D is a photograph of liver tissue from the group having cirrhosis that was administered with 30 mg/kg of oltipraz, 3 times a week for 4 weeks.

TABLE 1
Effect of Oltipraz on Cirrhosis

| Group | Fibrosis Scores | Knodell Scores |
|---|---|---|
| Control | 0 | 0 |
| Group with cirrhosis | 3.8 ± 0.2 | 14.0 ± 0.8 |
| Group with cirrhosis + | 2.9 ± 0.4 | 8.7 ± 1.1** |
| Oltipraz 15 mg/kg | | |
| Group with cirrhosis + | 2.8 ± 1.1* | 6.8 ± 2.5** |
| Oltipraz 30 mg/kg | | |
| Group with fibrosis | 2.2 ± 0.5^{a} | 6.0 ± 1.2^{a} |
| Group with fibrosis + | 2.8 ± 0.4 | 7.0 ± 1.2 |
| Oltipraz 15 mg/kg | | |
| Group with fibrosis + | 1.0 ± 0.0** | 2.6 ± 0.4* |
| Oltipraz 30 mg/kg | | |

Each value is represented by the average ± standard deviation. The number of animals used was 5 to 10. The significance of each group is determined by the paired Student's t-test. The significance is indicated by * p<0.05, ** p<0.01 compared to rats with cirrhosis or liver fibrosis. Rats with liver fibrosis had lower Knodell scores than rats with cirrhosis (a, p<0.05). Degree of fibrosis 0=Normal, 1=Presence of weak fibrous tissue, 2=Presence of moderate fibrous tissue, 3=Presence of obvious fibrous tissue, 4=Evidence of severe fibrosis. Sum of values from periportal bridging (Greatest=10), intralobular cell loss (Greatest=4), portal inflammation (Greatest=4), and fibrosis (Greatest=4) yields the Knodell score.

### Example 3

### Blood Biochemical Parameters of Animals with Liver Cirrhosis

Compared to normal animals, rats with cirrhosis showed increased activity of alanine aminotransferase (ALT) and aspartate aminotransferase (AST), by 3 to 4 times each. When the rats were administered with 15 mg/kg of oltipraz, 3 times a week for 4 weeks, the ALT and AST activity in the blood plasma was reduced, and when administered with 30 mg/kg, approximately 70% of the AST value lowered, showing a statistical significance (Table 2).

The amount of bilirubin in blood plasma is an indicator of the liver's capacity. As a result of oltipraz administration in cirrhotic rats, the amount of bilirubin, produced as an effect of cirrhosis, tended to be reduced. The total cholesterol level in blood plasma did not show noticeable change in cirrhotic rats or cirrhotic rats treated with oltipraz (Table 2).

TABLE 2
ALT, AST, Bilirubin and Cholesterol in Blood Plasma

| Group | ALT | AST | Bilirubin | Cholesterol |
|---|---|---|---|---|
| Control | 55 ± 4 | 141 ± 18 | 1.1 ± 0.1 | 97 ± 6 |
| Group with cirrhosis | 138 ± 14* | 275 ± 36* | 2.7 ± 0.8 | 152 ± 30 |
| Group with | 124 ± 47 | 206 ± 24 | 1.8 ± 0.4 | 116 ± 9 |
| cirrhosis + | | | | |
| Oltipraz 15 mg/kg | | | | |
| Group with | 110 ± 39 | 185 ± 22^{#} | 1.4 ± 0.2 | 104 ± 7 |
| cirrhosis + | | | | |
| Oltipraz 30 mg/kg | | | | |

Each value is represented by the average ± standard deviation. The number of animals used was 8 to 11. The significance of each group is determined by the paired Student's t-test. The significance is indicated by * p<0.05, compared to control and # p<0.05 compared to rats with cirrhosis.

### Example 4

### Effect of Oltipraz on Plasma Albumin Content and Ascites Formation

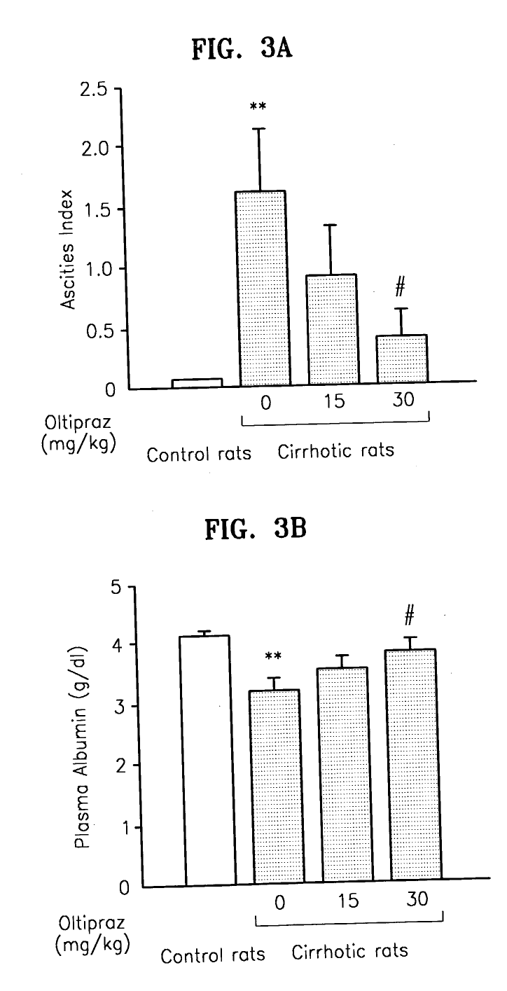

Another representative clinical symptom of cirrhosis is the accumulation of ascites. When the formation of ascites in 10 mode cirrhotic rats was examined by using the ascites formation index of 0=No visible ascites, 1=Presence of small amount of ascites between organs, 2=Noticeable flow of accumulated ascites after abdominal incision visible to the naked eye and 3=Noticeable eruption of the accumulated ascites after abdominal incision, the value was 1.7 for cirrhotic rats. After administering 15 mg/kg and 30 mg/kg of oltipraz, the value dropped to 0.9 and 0.4, respectively (FIG. 3). The significance is indicated by ** p<0.01, compared to control and # p<0.05 compared to rats with cirrhosis.

Ascites are formed when the synthesis of the blood plasma protein (especially albumin) is reduced in the liver tissues, bringing a disturbance in maintaining the equilibrium of the osmotic pressure in the blood. The amount of albumin in the blood plasma of cirrhotic rats decreased considerably. However, after the administration of 30 mg/kg of oltipraz, 3 times a week for 4 weeks, normal albumin content was recovered (FIG. 3b). The significance is indicated by ** p<0.01, compared to control and # p<0.05 compared to rats with cirrhosis.

### Example 5

### Effect of Oltipraz on Regeneration of Liver Tissue in Cirrhotic Rats

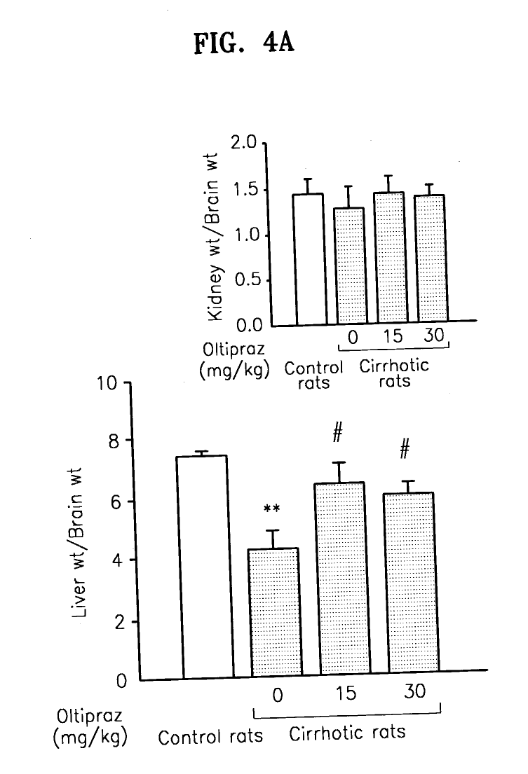

Cirrhosis is not only responsible for diminishing of a liver's function, but also for atrophy of the liver tissues. When the liver weights of 10 cirrhotic rats were examined, the weights were reduced to approximately 56% of normal liver weights. After the administration of 15 mg/kg and 30 mg/kg of oltipraz, 3 times a week for 4 weeks, the liver weights were recovered almost to the normal weights (FIG. 4a). On the other hand, the kidney weights did not show noticeable change (top of FIG. 4a). Since weight loss accompanies cirrhosis, in order to standardize the test results, the weight of the brain, which is normally not affected by cirrhosis, was used as the comparative weight to obtain the changes in the weights. The significance is indicated by ** p<0.01, compared to control and # p<0.05 compared to rats with cirrhosis.

After the administration of 30 mg/kg of oltipraz to cirrhotic rats, 3 times a week for 4 weeks, the division of the liver cells was observed with a microscope (FIG. 4b). The left side of FIG. 4b is a photograph of liver tissue, obtained after administration of oltipraz to cirrhotic rats, taken following Masson's trichrome staining. The photograph clearly shows the dividing cells, which is very rare in normal or cirrhotic liver tissues. Even when nuclear fast red staining was used, which selectively stains each nucleus, the cells under division and migration of the chromosomes were observed in the cirrhotic rats administered with oltipraz (Right side of FIG. 4b).

PCNA immunochemical staining method is often used to test the proliferation of the cells in animal models. PCNA is a stable cell-cycle nuclear protein (36 kDa), which is expressed in the late G1 and S phases of the cell cycle, and serves as an excellent marker for proliferating cells (Kawamura K, Kobayashi Y, Tanaka T, Ikeda R, Fujikawa-Yamamoto K, Suzuki K. Intranuclear localization of proliferating cell nuclear antigen during the cell cycle in renal cell carcinoma. 2000 Anal Quant Cytol Histol 22, 107-113.).

PCNA immunochemical analysis is done with a specific antibody for PCNA (Santa Cruz Biotech). Analysis was carried out by indirect Avidin-Biotin-Alkaline-Phosphatase technique, according to the protocol provided by the manufacturer (InnoGenex). Paraffinized slices of liver tissues from the control, cirrhotic rat and cirrhotic rat that had been administered with 30 mg/kg of oltipraz, for 3 times a week for 4 weeks, were placed on slides, paraffin was removed, and hydrated at room temperature. By using blocking serum, nonspecific antibody bindings were prevented. Then, in a humidified chamber, the slices were incubated with antibodies for 30 minutes at room temperature. After the incubation, Phosphate Buffered Saline (PBS) with 0.1% Tween-20 was used to rinse the slides. The slices were subjected to biotinylated 2^{nd} antibodies and reacted for 5 minutes at 37° C. followed by additional reaction for 5 minutes at 37° C. with streptavidin-conjugated alkaline phosphatase added. Then, 5-bromo-4-chloro-3-indolyl-phosphate (BCIP) and nitro-blue-tetrazolium (NBT) were used as the phosphatase substrate to be incubated with the slices on the slide until proper colors became visible. Afterwards, the slices were again stained with nuclear fast red.

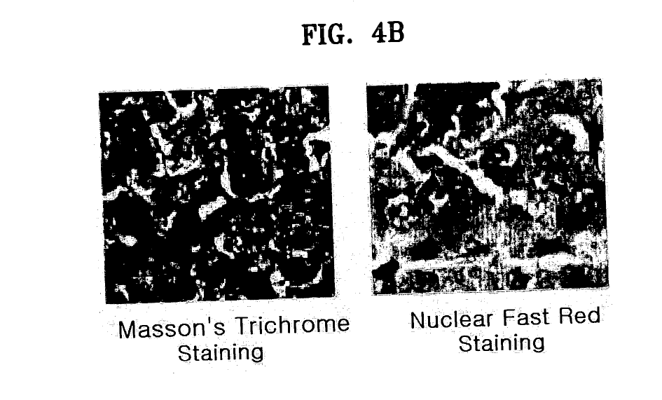

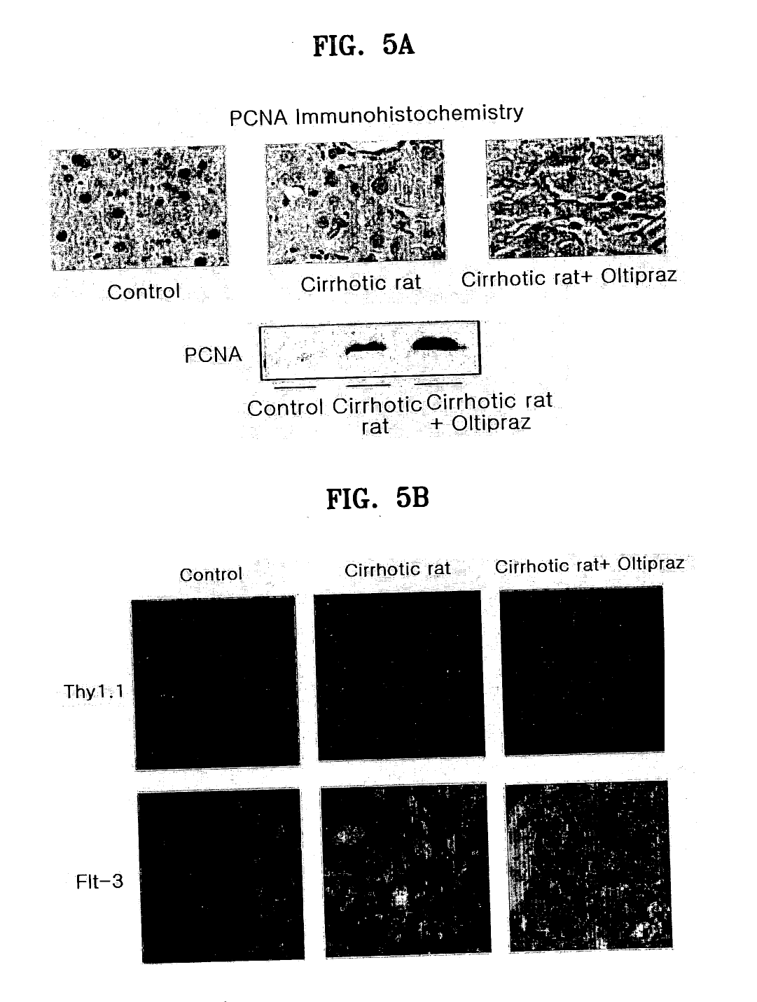

The result of such PCNA immunochemical staining method demonstrated that control animals did not show any cells with PCNA, but cirrhotic rats showed positive PCNA reaction near the fibers of blood vessels. In oltipraz administered cirrhotic rats, cells with PCNA were widely observed throughout the test sample. Compared to the sample from cirrhotic rat without oltipraz administration, the occurrence of PCNA was approximately twice more frequent in oltipraz administered rats (FIG. 5a).

Nuclear fractions of the liver from the control rats, cirrhotic rats and oltipraz administered (30 mg/kg, 3 times a week for 4 weeks) cirrhotic rats were dissolved in a diluting solution containing sodium dodecyl sulfate (SDS) to form a sample and stored at −70° C. After SDS-polyacrylamide gel electrophoresis, immunoblot analysis was carried out. The sample was fractionated by using 12% gel electrophoresis and electrically transferred to nitrocellulose membrane. The nitrocellulose membrane was incubated with polyclonal mouse anti-PCNA antibody (1:1000) (Santa Cruz Biotech) and then incubated again with horseradish peroxidase-conjugated secondary antibody. Lastly, ECL chemiluminescence kit manufactured by Amersham company was used to develop the band.

Even when western blot analysis was employed to study the expression of PCNA, there was a significant increase in the expression of PCNA, as evidenced by increase in the band intensity of the 36 kDa PCNA in the liver tissues of oltipraz administered cirrhotic rats than control rats or cirrohotic rats without treatment.

The cells, which regenerate to liver tissues, are known to originate from stem cells. In the present invention, Thy1.1 and Flt-3 (Santa Cruz Biotech), specific marker proteins of stem cells, were stained using the staining method similar to that of the PCNA to study the distribution of stem cells during cirrhosis. In cirrhotic rats, many cells containing Thy1.1 and Flt-3 were observed, but no such cells were observed in the control rats (FIG. 5b). In oltipraz administered (30 mg/kg, 3 times a week for 4 weeks) cirrhotic rats, there was a considerable decline in the number of cells containing Thy1.1 and Flt-3, compared to the cirrhotic rats without treatment. Such result is considered to be the result of the effect of oltipraz in converting the undifferentiated stem cells into differentiated liver cells.

### Example 6

### Effect of Oltipraz on the Expression of c-Met Suppressed by Liver Cirrhosis

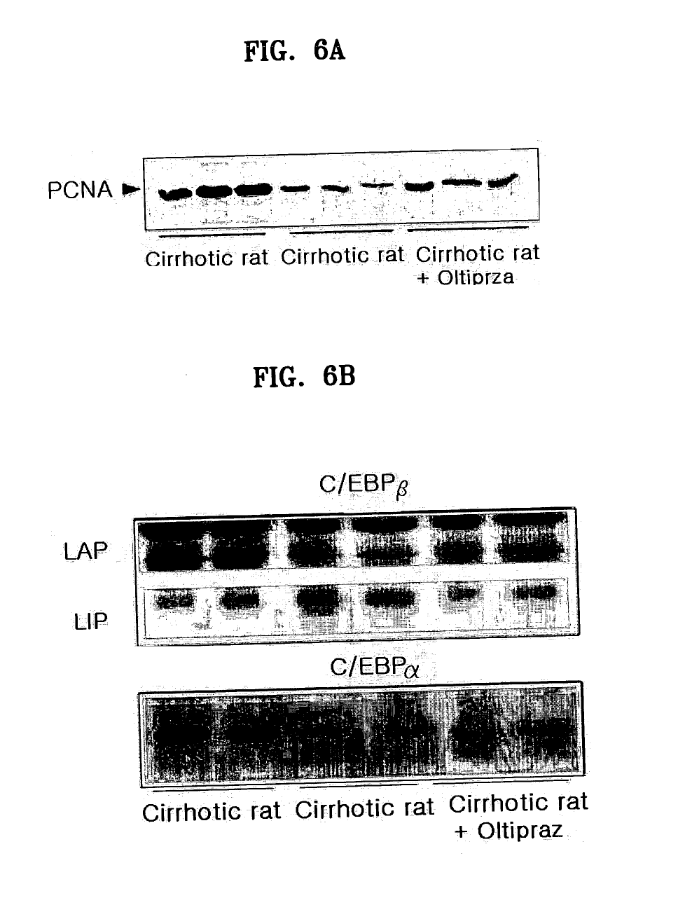

c-Met is a Hepatocyte Growth Factor (HGF) receptor, applicable in proliferation and differentiation of the liver cells. The expression of c-Met declines with cirrhosis (FIG. 6a). When c-Met is not appropriately present, even with the presence of HGF, liver tissues will not form.

In oltipraz administered (30 mg/kg, 3 times a week for 4 weeks) cirrhotic rats, the expression of c-Met was noticeably greater than un-administered rats (FIG. 6a). Such result is consistent with the notion that oltipraz regenerates liver tissues that have undergone cirrhosis.

### Example 7

### Effect of Oltipraz on the Translocation of C/EBP to the Nucleus

Related in the proliferation of the liver cells, important transcription factors are C/EBP-β and C/EBP-α, belonging in the C/EBP family. Among the two, C/EBP-β is considered to be more important in the proliferation of the liver cells. When C/EBP-β gene is removed from a mouse, the restoration of the liver size after a partial hepatectomy is significantly decreased. (Greenbaum L E, Li W, Cressman D E, Peng Y, Ciliberto G, Poli V, Taub R., CCAAT/enhancer-binding protein beta is required for normal hepatocyte proliferation in mice after partial hepatectomy. J Clin Invest. (1998) 102:996-1007).

With the importance of C/EBP in the regulation of liver regeneration in mind, the expression of C/EBP-β and C/EBP-α in oltipraz administered (30 mg/kg, 3 times a week for 4 weeks) cirrhotic rats were examined. The expression of C/EBP-β, which declined in a cirrhotic rat, increased in oltipraz administered cirrhotic rats. After cirrhotic rat was administered with 30 mg/kg of oltipraz, 3 times a week for 4 weeks, the appearance of liver-enriched inhibitory protein (LIP), which is an isoform of C/EBP-β, and the manifestation of which increases in cirrhotic rats, had almost completely disappeared. C/EBP-α, although manifested in control rats, is noticeably reduced in a cirrhotic rat. However, after cirrhotic rat was administered with 30 mg/kg of oltipraz, 3 times a week for 4 weeks, the expression of C/EBP-α was noticeably recovered.

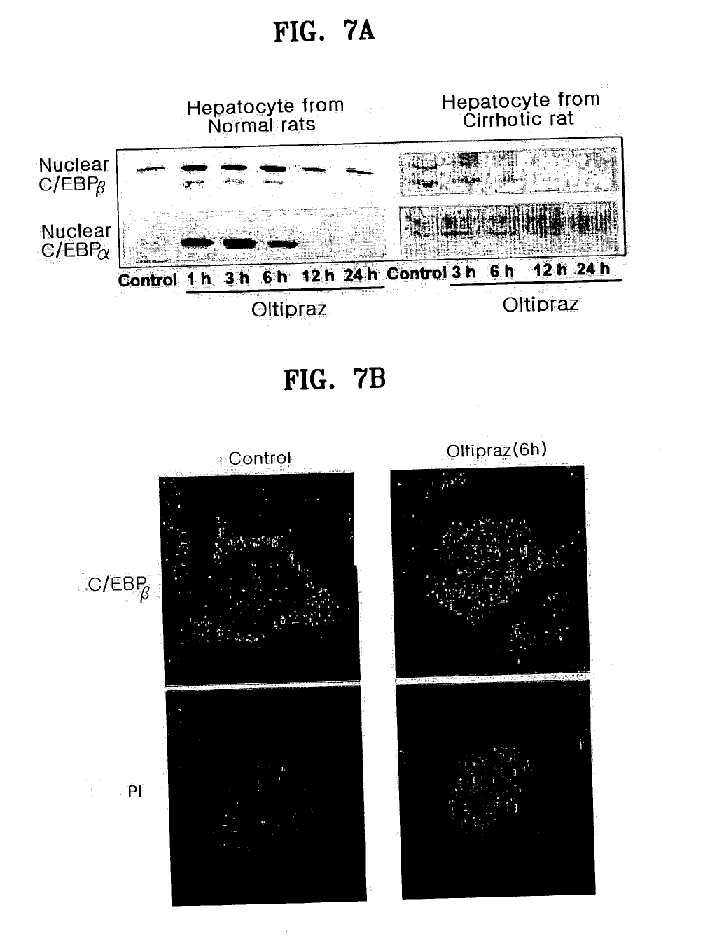

Because of the evident activity of C/EBP found in the oltipraz administered cirrhotic rats, next test quantified the amount of C/EBP translocated into the nucleus in primary cultured hepatocytes by utilizing western blot method to test the direct effect of oltipraz on the C/EBP activity at liver cells. When the primary cultured hepatocytes were incubated with oltipraz at the concentration of 30 μM, the amount of C/EBP-β and C/EBP-α in the nucleus of the liver cells gradually increased (FIG. 7a). However, when the hepatocytes isolated from cirrhotic rats were incubated with oltipraz, no significant change was observed. Such result demonstrated that oltipraz directly triggers C/EBP activity.

Afterwards, to study whether oltipraz promotes translocation of C/EBP-β to the nucleus, rat hepatocytes were incubated with oltipraz at the concentration of 30 μM for 6 hours. According to the immunocytochemical analysis, oltipraz clearly promoted translocation of C/EBP-β to the nucleus (FIG. 7b).

Therefore, the regeneration of the liver tissues by oltipraz is accompanied by C/EBP activation.

As demonstrated in the foregoing, oltipraz can effectively regenerate cirrhotic liver tissues, and the pharmaceutical composition of the present invention is highly effective in regenerating the liver tissues undergone cirrhosis and curing cirrhotic liver.

### Preparation Example 1

| | |
|---|---|
| Oltipraz | 25 mg |
| Lactose | 50 mg |
| Starch | 10 mg |
| Magnesium stearate | Proper amount |

The above components are mixed and a tablet is prepared by a conventional tablet preparation method.

### Preparation Example 2

| | | |
|---|---|---|
| Oltipraz | 100 | mg |
| Lactose | 50 | mg |
| Starch | 10 | mg |

| | |
|---|---|
| Magnesium stearate | Proper amount |

The above components are mixed and a tablet is prepared by a conventional tablet preparation method.

### Preparation Example 3

| | | |
|---|---|---|
| Oltipraz | 250 | mg |
| Lactose | 50 | mg |
| Starch | 10 | mg |

| | |
|---|---|
| Magnesium stearate | Proper amount |

The above components are mixed and a tablet is prepared by a conventional tablet preparation method.

### Preparation Example 4

| | | |
|---|---|---|
| Oltipraz | 25 | mg |
| Lactose | 30 | mg |
| Starch | 28 | mg |
| Talc | 2 | mg |

| | |
|---|---|
| Magnesium stearate | Proper amount |

The above components are mixed and a gelatin hard capsule is prepared by a conventional gelatin hard capsule preparation method.

### Preparation Example 5

| | | |
|---|---|---|
| Oltipraz | 100 | mg |
| Lactose | 30 | mg |
| Starch | 28 | mg |
| Talc | 2 | mg |

| | |
|---|---|
| Magnesium stearate | Proper amount |

The above components are mixed and a gelatin hard capsule is prepared by a conventional gelatin hard capsule preparation method.

### Preparation Example 6

| | | |
|---|---|---|
| Oltipraz | 250 | mg |
| Isomerized sugar | 10 | g |
| Sugar | 30 | mg |
| Sodium CMC | 100 | mg |

| | |
|---|---|
| Lemon Flavor | Proper amount |
| (add distilled water | |
| for the total volume of 100 ml) | |

A suspension is prepared with the above components according to conventional suspension preparation methods. A 100 ml brown bottle is filled with the suspension and

### Preparation Example 7

| | | |
|---|---|---|
| Oltipraz | 500 | mg |
| Isomerized sugar | 20 | g |
| Sugar | 20 | mg |
| Sodium arginate | 100 | mg |

| | |
|---|---|
| Orange Flavor | Proper amount |
| (add distilled water | |
| for the total volume of 100 ml) | |

A suspension is prepared with the above components according to conventional suspension preparation methods. A 100 ml brown bottle is filled with the suspension and sterilized.

### Preparation Example 8

| | | |
|---|---|---|
| Oltipraz | 250 | mg |
| Lactose | 30 | mg |
| Starch | 20 | mg |

| | |
|---|---|
| Magnesium stearate | Proper amount |

The above components are mixed and filled in a polyethylene coated envelope and sealed to prepare a powder.

### Preparation Example 9

1 Soft capsule containing

| | | |
|---|---|---|
| Oltipraz | 100 | mg |
| Polyethylene glycol | 400 | mg |
| Concentrated glycerin | 55 | mg |
| Distilled water | 35 | mg |

Polyethylene glycol is mixed with concentrated glycerin, and then distilled water is added. Maintaining the mixture at 60° C., oltipraz is added to the mixture. The mixture is stirred at approximately 1,500 rpm. After the mixture has been mixed uniformly, the mixture is cooled at room temperature under slow stirring. Air bubbles are removed with a vacuum pump, leaving the contents of the soft capsule.

The soft capsule membrane is manufactured according to conventional preparation methods using a widely known soft gelatin-plasticizer formula containing gelatin 132 mg, concentrated glycerin 52 mg, 70% disorbitol solution 6 mg per capsule, a proper amount of ethyl vanillin flavoring agent, and carnauba wax as the coating agent.

The pharmaceutical composition comprising oltipraz presented in the present invention is clinically useful in promoting regeneration of liver tissues of a cirrhotic liver and the composition exhibits effective treatment of cirrhosis.

What is claimed is:

1. A pharmaceutical composition for regeneration of liver tissues to treat liver cirrhosis comprising 5-(2-pyrazinyl)-4-methyl-1,2-dithiol-3-thione (oltipraz) and a pharmaceutically acceptable excipient.

2. A pharmaceutical composition according to claim 1, wherein the composition is formulated in a form selected from the group consisting of a capsule, a tablet, a soft capsule, a suspension, a syrup, an injection, and a powder.

3. A pharmaceutical composition according to claim 1, wherein the composition is formulated in a form for oral administration

4. A method of regenerating liver tissues to treat liver cirrhosis comprising administering 5-(2-pyrazinyl)-4-methyl-1,2-dithiol-3-thione to a mammal.
